(19) United States
(12) Patent Application Publication
(10) Pub. No.: US 2017/0339484 A1
(43) Pub. Date: Nov. 23, 2017

(54) SMART AUDIO HEADPHONE SYSTEM

(71) Applicant: NGOGGLE INC., San Diego, CA (US)

(72) Inventor: Revyn Kim, San Diego, CA (US)

(21) Appl. No.: 15/522,730

(22) PCT Filed: Nov. 2, 2015
(86) PCT No.: PCT/US15/58647

§ 371 Date: Apr. 27, 2017

Related U.S. Application Data
(60) Provisional application No. 62/074,042, filed on Nov. 2, 2014.

(51) Int. Cl. H04R 1/10; A61B 5/00; A61B 5/16; A61B 5/0478

(52) U.S. Cl. CPC H04R 1/1041; H04R 1/105; H04R 1/1008; A61B 5/0478; A61B 5/6815; A61B 5/165; A61B 5/6822

(57) ABSTRACT

The present invention relates to SMART headphones. More particularly, the present invention relates to SMART audio headphones system adapted to modulate personal playlists that adapt to a users preferences, particularly to their state of mind and/or emotions.

### TECHNICAL FIELD

The present invention relates to SMART headphones. More particularly, the present invention relates to SMART audio headphones system adapted to modulate personal playlists that adapt to a user's preferences, particularly to their state of mind and/or emotions.

### DESCRIPTION OF THE RELATED ART

ZEN TUNES is an iPhone app that analyses the brainwaves emitted when listening to music and produces a music chart based on the listeners “relax” and “focus” state. ZEN TUNES provides “awareness” by tagging the listeners' brainwaves to the music they listen too.

An extension of this is seen with the Mico headphone, which applies a single EEG sensor on the forehead of the listener. The mico headphone detects brainwaves through the sensor on the forehead. The mico app (ZEN TUNES) then analyzes the user's condition of the brain, and searches for music that matches from the mico music data base, and plays the selection that fits the user's status.

Method And System For Analysing Sound, U.S. Patent Application 20140307878.

The present invention relates to a method and system for analysing audio (eg. music) tracks. A predictive model of the neuro-physiological functioning and response to sounds by one or more of the human lower cortical, limbic and subcortical regions in the brain is described. Sounds are analysed so that appropriate sounds can be selected and played to a listener in order to stimulate and/or manipulate neuro-physiological arousal in that listener. The method and system are particularly applicable to applications harnessing a biofeedback resource.

Audio headset with bio-signal sensors, U.S. Pat. No. 8,781,570

Ruo-Nan Duan, Xiao-Wei Wang, Bao-Liang Lu. EEG-Based Emotion Recognition in Listening Music by Using Support Vector Machine and Linear Dynamic System. Neural Information Processing: Lecture Notes in Computer Science Volume 7666, 2012, pp 468-475.

### SUMMARY

The present invention is described as a system that includes an audio headphone having one or more audio speakers and one or more bio-signal sensors that can learn and detect a user's emotions, moods and/or preferences (EMP) in relationship to music that is being played to the user, a method of collection and analysis of the bio-signals collected over time catalogued by user listener and song title, a method of identifying and relating attributes of a piece of music to specific moods and/or emotions, and a method for adaptively and automatically selecting music based on learned emotions, moods and/or preferences to a specific user.

### BRIEF DESCRIPTION OF THE DRAWINGS

Embodiments of the present disclosure will be better appreciated by reference to the drawings wherein:

FIG. 1 is an illustration of a SMART audio headphone system;

FIG. 2 is an illustration of a SMART audio headphone system;

FIG. 3 is an illustration of a SMART audio headphone system;

FIG. 4 is an illustration of a SMART audio earphone system with sensors placed on headband;

FIG. 5 is an illustration of a SMART audio earphone system with contactless sensors placed on headband;

FIG. 6 is an illustration of a SMART audio in-ear headphone unit;

FIG. 7 is an illustration of a SMART audio earphone system with bio-sensors that circumvent the neck of the user;

FIG. 8 is an illustration of a SMART audio headphone collecting EEG and ECG bio-signals;

FIG. 9 depicts the flowchart for learning emotions, moods and/or preferences (EMP);

FIG. 10 depicts the flowchart for a process to automatically and adaptively select music that employs a machine classifier to learn and match selective physiological signals to appropriate music;

FIG. 11 depicts the process for a user to initiate the training of a system to learn EMP;

FIG. 12 depicts a flowchart for a process to learn the attributes of music associated with an EMP of a user;

FIG. 13 depicts data stores accessed by the system;

FIG. 14 is a block diagram illustrating a computer system that is able to perform the methods of FIGS. 8-10;

FIG. 15 is a schematic drawing illustrating devices and computer systems accessing music databases;

FIG. 16 is an emotion chart.

### DETAILED DESCRIPTION OF THE EMBODIMENTS

A detailed description of one or more embodiments of the invention is provided below along with accompanying figures that illustrate the principles of the invention. The invention is described in connection with such embodiments, but the invention is not limited to any embodiment and encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

Accordingly, reference throughout this specification to “one embodiment,” “an embodiment,” “certain embodiment”, or similar language means that a particular feature, structure, or characteristic described in connection with the embodiment is included in at least one contemplation or embodiment of the invention, and expressly does not mean in all embodiments. Thus, appearances of the phrases “in one embodiment,” “in an embodiment,” and similar language throughout this specification may, but do not necessarily, all refer to the same embodiment, but mean “one or more but not all embodiments” unless expressly specified otherwise. In addition, various embodiments of the invention are described with various modular features. The features described are modular and can be used in any embodiment, not necessarily in that particular described embodiment, or at all. The terms “including,” “comprising,” “having,” and variations thereof mean “including but not limited to,” unless expressly specified otherwise. An enumerated listing of items does not imply that any or all of the items are mutually exclusive, unless expressly specified otherwise. The terms “a,” “an,” and “the” also refer to “one or more” unless expressly specified otherwise.

As will be appreciated by one skilled in the art, aspects of the present invention may be embodied as a system, device, apparatus, method, or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, certain aspects of the present invention may take the form of an electronic device having therein a computer program product embodied in one or more computer readable medium(s) having computer readable program code embodied thereon and/or on client devices.

Furthermore, the described features, structures, or characteristics of the embodiments may be combined in any suitable manner. In the following description, numerous specific details are provided, such as examples of programming, software modules, user selections, network transactions, database queries, database structures, hardware modules, hardware circuits, hardware chips, devices, apparatus, etc., to provide a thorough understanding of embodiments. One skilled in the relevant art will recognize, however, that embodiments may be practiced without one or more of the specific details, or with other methods, components, materials, and so forth. In other instances, well-known structures, materials, or operations are not shown or described in detail to avoid obscuring aspects of an embodiment.

In one embodiment of the invention, the invention described herein is particularly applicable to a SMART audio headphone system to adaptively and automatically select and listen to music based on learned emotions, moods and/or preferences (EMP) of the user. The system comprises an audio headphone (aka headset, headphone, earbud, earphones, or earcans) having one or more audio speakers and one or more bio-signal sensors (e.g., a over-the-ear or earbud headphone with EEG sensors (e.g., electrodes)) that adaptively extracts and classifies one or more bio-signal to learn a user's emotions, moods and/or preferences, and selects music that matches the emotion, mood and/or preference of the user, and it is in this context that the device will be described. Music refers to vocal, instrumental, or mechanical sounds that may or may not have rhythm, melody, or harmony (e.g., a tune, jingle, song, noise music, etc.), which may include the entire composition or parts thereof. The specific use of these terms, e.g., song, tune, musical piece, composition should not be interpreted to limit the invention as these terms are used interchangeably and as examples of the broader concept, audio sounds.

In an alternate or additional embodiment, the audio headphone system comprises a learning mechanism to classify attributes of music based on one or multiple user's preferences, moods and/or emotions. For example, music may be automatically classified and labeled based on a person's personal preferences for music, emotion, or mood, or based on a person's personal classification (e.g., genre, activity, intended use, etc.).

As described herein, emotions, moods and/or preferences are based on physiological or behavioral representations of an emotion, mood and/or preferences. For purposes of this innovation, any set of emotion, mood or preference definitions and hierarchies can be used which is recognized as capturing at least a human emotion or preference element, including those described in the field of art/entertainment, marketing, psychology, or those newly derived by the invention herein. For example, preferences can be as simple as personal likes and dislikes and indifference; or much more complex for example, the emotion annotation and representation language (EARL) proposed by the Human-Machine Interaction Network on Emotion (HUMAINE):

negative and forceful (e.g., anger, annoyance, contempt, disgust, irritation), negative and not in control (e.g., anxiety, embarrassment, fear, helplessness, powerlessness, worry), negative thoughts (e.g., doubt, envy, frustration, guilt, shame), negative and passive (e.g., boredom, despair, disappointment, hurt, sadness), agitation (e.g., stress, shock, tension), positive and lively (e.g., amusement, delight, elation, excitement, happiness, joy, pleasure), caring (e.g., affection, empathy, friendliness, love), positive thoughts (e.g., courage, hope, pride, satisfaction, trust, quiet positive (e.g., calmness, contentment, relaxation, relief, serenity), reactive (e.g., interest, politeness, surprise).

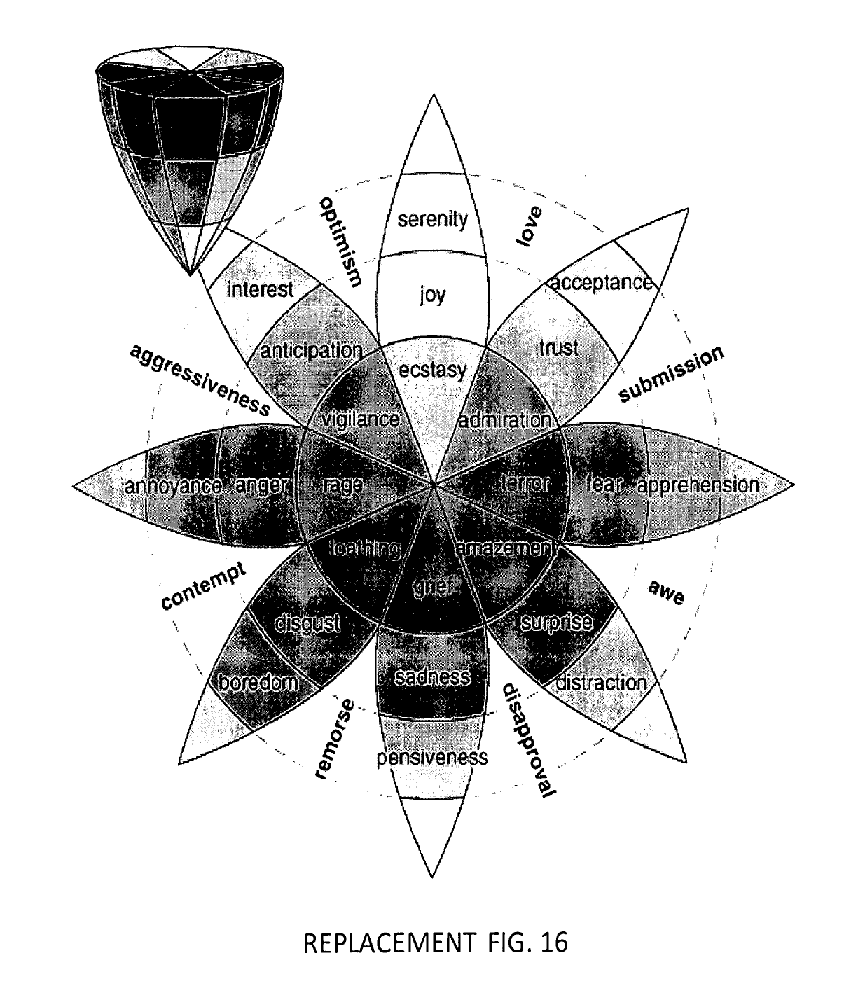

Other systems include Robert Plutchik's defined eight primary emotions of: anger, fear, sadness, disgust, surprise, anticipation, trust, and joy [Plutchik, R.: Emotions and life: perspectives from psychology, biology, and evolution. American Psychological Association, Washington, DC, 1st edn. (2003)]; or, Paul Ekman's list of basic emotions are: anger, fear, sadness, happiness, disgust and surprise, which expanded into amusement, contempt, contentment, embarrassment, excitement, guilt, pride in achievement, relief, satisfaction, sensory pleasure, and shame [Ekman, P.: Basic emotions. In: Dalgleish, T., Power, M. (eds.) Handbook of Cognition and Emotion. Wiley, New York (1999)]. Other emotion systems are also contemplated; see for example, FIG. 16. Particularly useful emotion sets include those utilized for entertainment, marketing or purchase behavior (See, e.g., Shrum L J (ed). The Psychology of Entertainment Media: Blurring the Lines between Entertainment and Persuasion. (Lawrence Erlbaum Associates, 2004); Bryant & Vorderer (eds). Psychology of Entertainment. (Routledge, 2006); Deutsch D (ed). The Psychology of Music, Third Edition (Cognition and Perception). (Academic Press, 2012).)

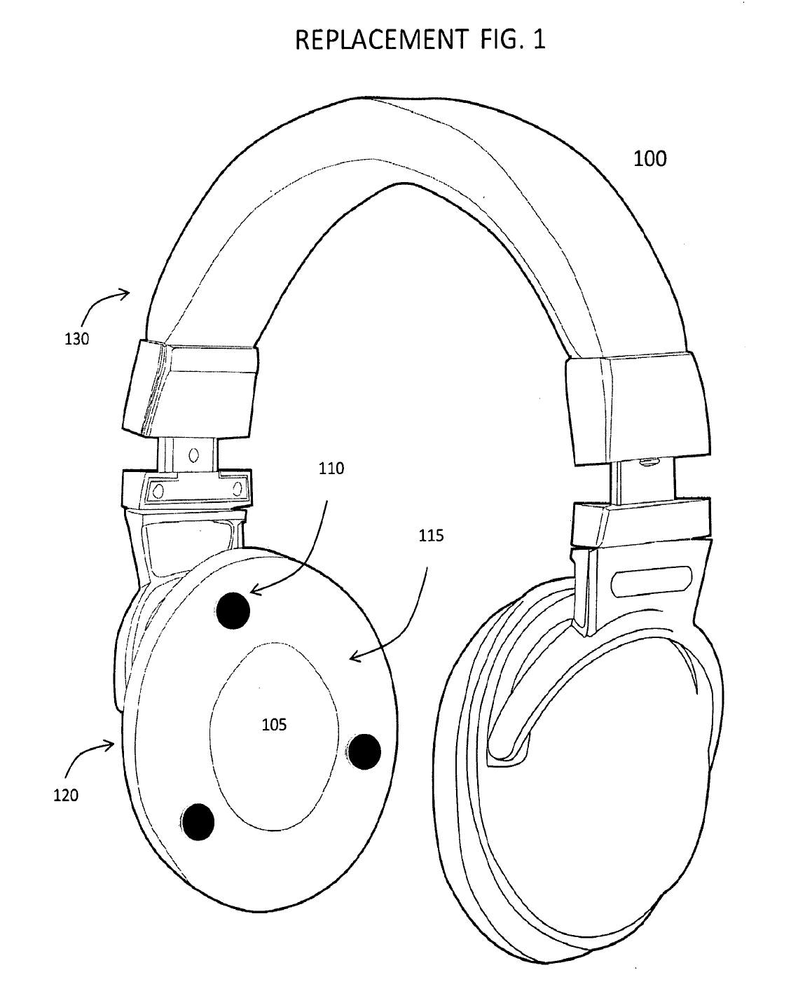

Embodiments of the present disclosure are illustrated in FIGS. 1-16.

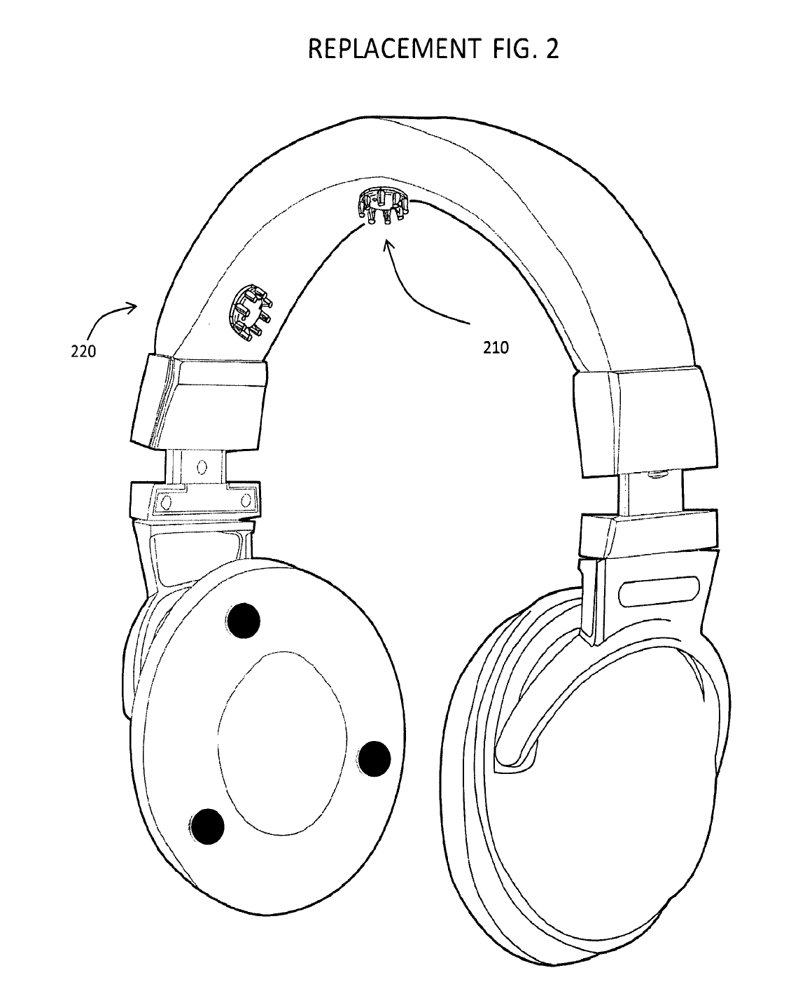

In one embodiment, the present disclosure is directed to a SMART audio electroencephalogram (EEG) headphone to measure brain electrical activity, comprising an audio headphone to support a plurality of electrodes in a configuration to acquire and monitor electroencephalogram (EEG) signals. FIG. 1 depicts one embodiment of a system 100 for a SMART audio headphone system. The system 100, in the depicted embodiment, includes an audio headphone module 100 configured to acquire one or more EEG signals, such as through an electrode or sensor 110. The electrodes 110 can be positioned to read an EEG signal from the skin of the user, such as for example the skin on the ear, surrounding the ear of the user, or along the hairline around the ear or on the neck. In an alternate or additional embodiment, as shown in FIG. 2, one or more sensors 210 can be placed along the headband 220 of the headphone to acquire and monitor EEG signals from the scalp, for example through electrode teeth that protrude through the hair to reach the skin. Headphone can be decorated or simple, or designed such to fit consumer trend.

Each electrode is electrically connected to electronic circuitry that can be configured to receive signals from the electrodes and provide an output to a processor. The electronic circuitry may be configured to perform at least some processing of the signals received from the electrodes. In some implementations electronic circuitry can be mounted on or housed within the headphone. In one embodiment, the EEG signal acquisition circuitry includes a processor, an analog signal processing unit, and an A/D (analog/digital) converter, but not limited, for example, filter and amplifier also can be included therein. In an alternate or additional embodiment, some processing of the signals may be performed by processors in a remote receiver on a separate device of the invention system, which could be on a separate client device such as a PC or mobile device or a separate computer on a web server via a network. In one embodiment, electronic circuitry includes components to modify or upgrade software, for example, wired or wireless components to enable programming modifications. Electronic circuitry also includes external interfaces such as electronic interfaces (e.g., ports), user interfaces (e.g., touch or touch-less controller, status interface such as an LED or similar screen/lights), and the like.

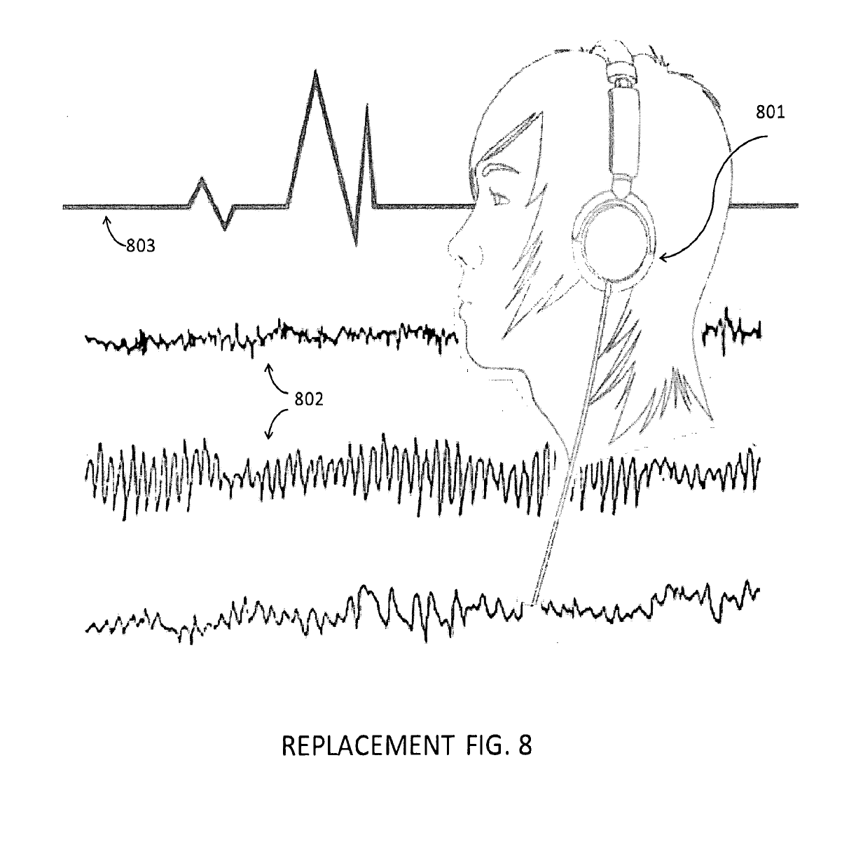

It will be appreciated that the device, for example, the audio headphone can be used with other types of sensors including other types of bio-signal sensors and/or other types of multimedia capabilities, such as audio/hearing bone conduction, motion sensors such as gyroscopes and accelerometers, headphone video head mounted display (e.g., video glasses with audio speakers) and/or 3D stereoscopic. Such bio-signals include those such as electrocardiogram (ECG/EKG), skin conductance (SC) or galvanic skin response (GSR), electromyography (EMG), respiration, pulse, electrooculography (EOG), pupillary dilation, eye tracking, facial emotion encoding, and reaction time devices, etc. and so on. An electrical bio-sensor can be used redundantly for multiple measurements such as a differential amplifier that measures the difference (e.g., EEG, ECG, EOG and/or EMG) and/or electrical resistance (e.g., GSR) between two electrodes attached to the skin. FIG. 8 shows a SMART audio headphone that measures both EEG and ECG. Sensors can be placed on the headband, on or inside of the earpieces of the headphone (and/or otherwise located in connection with the headphone) or positioned otherwise conducive to measuring the desired information.

FIG. 1 shows one embodiment of a speaker headset, although in some embodiments, the headphone is a mono-headset, in which there is only one earpiece instead of two earpieces. The headset 100 contains electrical components and structures (not illustrated) encased in the headband 130 and earpiece 120 to protect the electrical components and provide a comfortable fit, while measuring electrical signals from the surface of the user's head. The headband 130 can house electronics (not illustrated) such as a battery and other electronic components (wireless transmitter, processor, etc.) with wires or leads to each electrode 110. Power can come from batteries within device or powered by an external device through wiring. In one embodiment, headset 100 is adapted and configured for positioning about a wearer's head, e.g., along the crown of the head. The earpiece 120 includes both audio speakers 105 and EEG sensors 110. The EEG sensors 110 can be placed on the earpiece 120 to provide direct contact with the skin surrounding the ear or on the ear. Earpads 115 may be utilized to support the placement of the electrodes 110. In one embodiment, the earpads 115 can be made of a elastomeric or flexible material (e.g., resilient or pliant material such as foam, rubber, plastic or other polymer, fabric, or silicon) and shaped to accommodate different users' head and ear shape and sizes, provide wearing comfort, while providing enough pressure and positioning of the electrodes to the skin to ensure proper contact. In one embodiment, electrodes are positioned by the arcuate shape of the headband holding the earpad in position against ear.

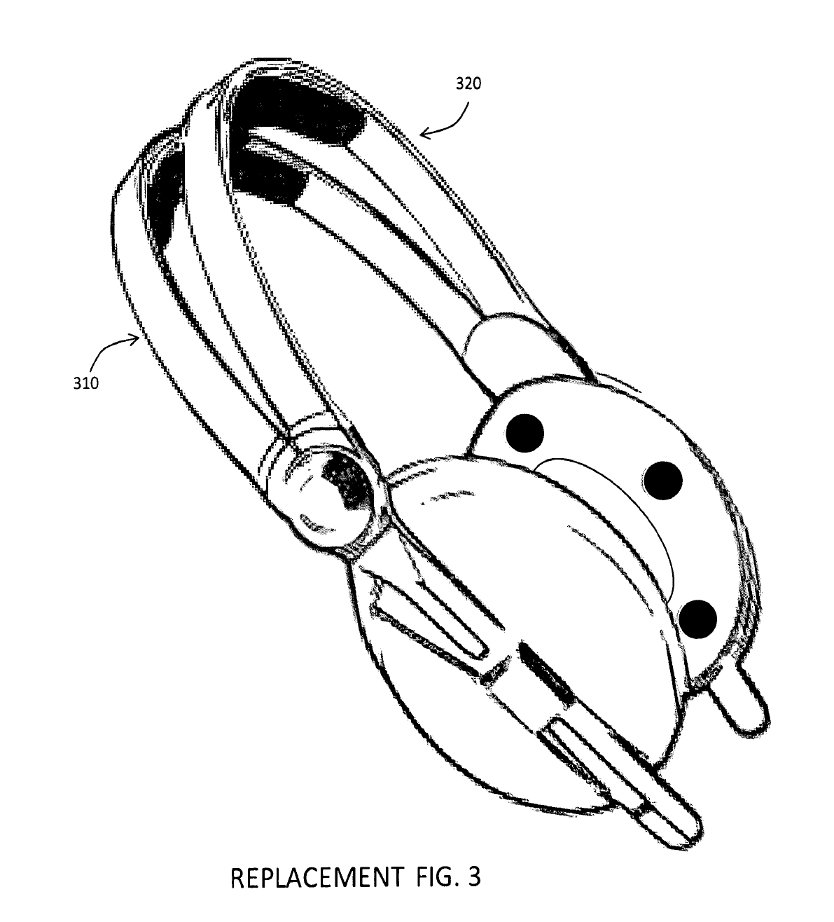

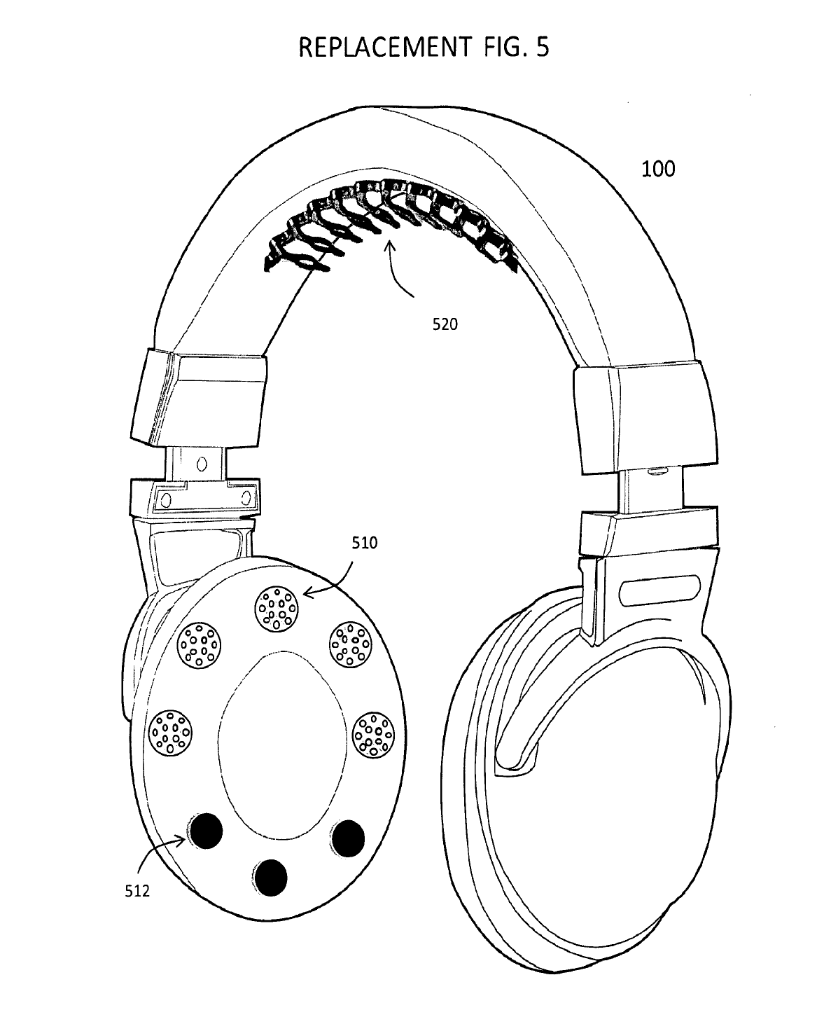

FIG. 2 shows one embodiment with a SMART audio headset having a headband that includes one or a plurality of electrode teeth or extenders 210 to provide contact or near contact with the scalp of a user. Teeth can circumnavigate headband to record EEG signals across, for example, the top of the head from ear to ear. Multiple headbands 310 and 320 can be used to measure different cross sections of the head (see, e.g., FIG. 3). Teeth can be permanently attached to headband or can be removable/replaceable, for example, plug-in sockets or male/female sockets. Each tooth can be of sufficient length to reach the scalp, spring-loaded or pliable/flexible to “give” upon contact with the scalp, or contactless to capture EEG signals without physical contact. Teeth 210 may have rounded outer surfaces to avoid trauma to the wearer's head, more preferably flanged tips to ensure safe consistent contact with scalp. Teeth 210 may be arranged about aperture or, alternatively, in one or more linear rows provided in spaced relation along headband. The teeth 210 may be made of fabric, polymeric, or metal materials that may provide additional structure, stiffness, or flexibility to the headband 210 to assist in placing the contacts 230 with the scalp of the user. The invention further contemplates electrodes for different location placements, for example, as shown in FIG. 5, teeth or extenders can be presented as teeth on a comb or barrette 520 attached or attachable on headband. For example, electrodes for the top of the head may encounter hair. Accordingly, electrodes on the ends of “teeth”, clips or springs may be utilized to reach the scalp of the head through the hair. Examples of such embodiments as well as other similar electrodes on headbands are discussed in U.S. patent application Ser. No. 13/899,515, entitled EEG Hair Band, incorporated herein by reference.

Any of a variety of electrodes known for use with EEG can be used with the present device. In one embodiment, the earpiece can comprise one electrode or multiple electrodes. In one embodiment, the earpiece can be entirely conductive. In yet another embodiment, one or more electrodes for use with the present device can be embedded or encompassed within or on the surface of an earpad made from a non-conducting material surrounding the conductive electrode unit. In yet another embodiment, electrodes can be etched or printed on to semi- or non-conductive surface. The non-conducting material such as fabric (including synthetic, natural, semi-synthetic and animal skin), can be used to separate/space each electrode, if more than one, or to localize the bio-signal to the point of contact. Electrode sensors utilized in the invention can either be entirely conductive, mixed or associated with or within non-conductive or semi-conductive material, or partially conductive such as on the tips of electrodes. For example, in certain embodiments, the conductive electrodes are woven with-in or with-out non-conductive material into a fabric, net, or mesh-like material to increase flexibility and comfort of the electrode or embedded or sewn into the fabric or other substrate of the head strap, or by other means. In one embodiment, the EEG sensors are dry electrodes or semi-dry electrodes. Electrode sensor material may be a metal such as stainless steel or copper, such as inert metals, like, gold, silver (silver/silver chloride), tin, tungsten, iridium oxide, palladium, and platinum, or carbon (e.g. graphene) or other conductive material, or combinations of the above, to acquire an electrical signal. The conductive material can further be a coating or integrated within the electrode, for example, mixed-in with other materials, e.g., graphene or metal mixed with rubber or silicone or polymers to result in the final electrode. The electrode can also be removable, including for example, a disposable conductive polymer or foam electrode. The electrode can be flexible, preshaped or rigid, or rigid within a larger flexible earpiece, and in any shape, for example, a sheet, rectangular, circular, or such other shape conducive to make contact with the wearer's skin. For example, electrode can have an outfacing conductive layer to make contact with the skin and an inner connection (under surface of earpiece) to connect to the electronic components of the invention. In some embodiments, the electrodes may be constructed using microfabrication technology to place numerous electrodes in an array configuration on a flexible substrate. In various embodiments the stimulating arrays comprise one or more biocompatible metals (e.g., gold, platinum, chromium, titanium, iridium, tungsten, and/or oxides and/or alloys thereof) disposed on a flexible material.

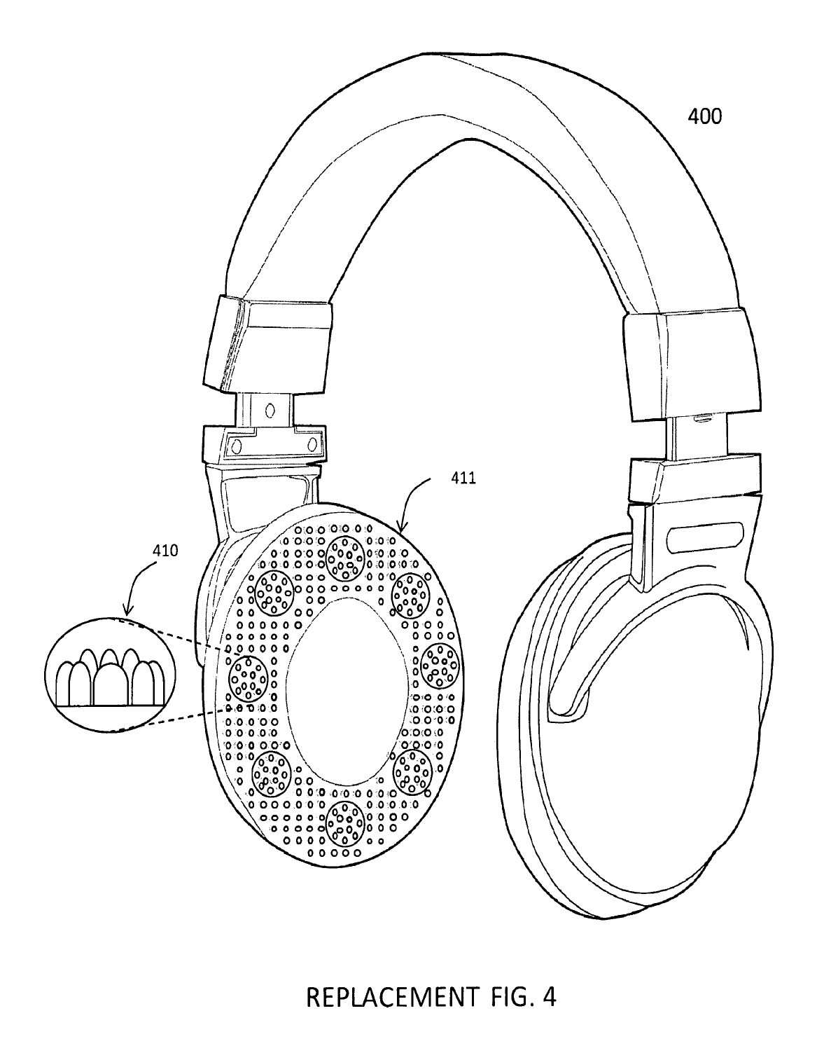

One example illustrated in FIG. 4 shows electrode teeth 410/411 that are redundantly placed on the earpiece of the device. Electrode teeth or electode bumpers 410/411 can be of varying sizes (e.g., widths and lengths), shapes (e.g., silo, linear waves or ridges, pyramidal), material, density, form-factors, and the like to acquire strongest signal and/or reduce noise, especially to minimize interference of the hair. FIG. 4 illustrates several independent electrodes 410 comprising conductive redundant bumpers in one electrode surrounded by an array 411 of independent bumpers 411 which may or may not be conductive. The independent bumper may used as one large electrode. FIG. 5 illustrates discrete placement of bumper electrodes 510 near hairline and non-bumper electrodes 512 on the lower portion of the earpiece where they may encounter less hair. In one embodiment, electrodes are made of foam or similar flexible material having conductive tips or conductive fiber to create robust individual connections without potential to irritate the skin of the user (e.g., “poking”). As reference and better understanding, without limitations, such material and design can be found in certain “massage” sandals that utilize bumpers to support the feet. Design of the bumper electrodes can incorporate factors that maximize connection (e.g., compressed contact, streamlined designed to part hair to reach scalp), reduce noise, increase durability, mitigate discomfort and/or increase comfort and ergonomics, and the like. For example, electrode bumpers can be surrounded by non-conductive bumpers made of durable material to protect the conductive bumpers that may use more flexible material, or in an array to minimize discomfort, and/or maximize durability of the electrodes.

The present invention contemplates different combinations and numbers of electrodes and electrode assemblies to be utilized. As to electrodes, the amount and arrangement thereof both can be varied corresponding to different demands, including allowable space, cost, utility and application. Thus, there is no limitation. The electrode assembly typically will have more than one electrode, for example, several or more electrode each corresponding to a separate electrode lead, although different numbers of electrodes are easily supported, in the range of 2-300 or more electrodes per each earpiece, for example. One or more electrodes can be connected by one lead as one redundant arrayed electrode, connected by several leads with each lead to a plurality of electrodes grouped for each group to record different signals (e.g., channels) or a single lead to each electrode that can be distinct and independent of other electrodes to create an array of distinct signals or channels.

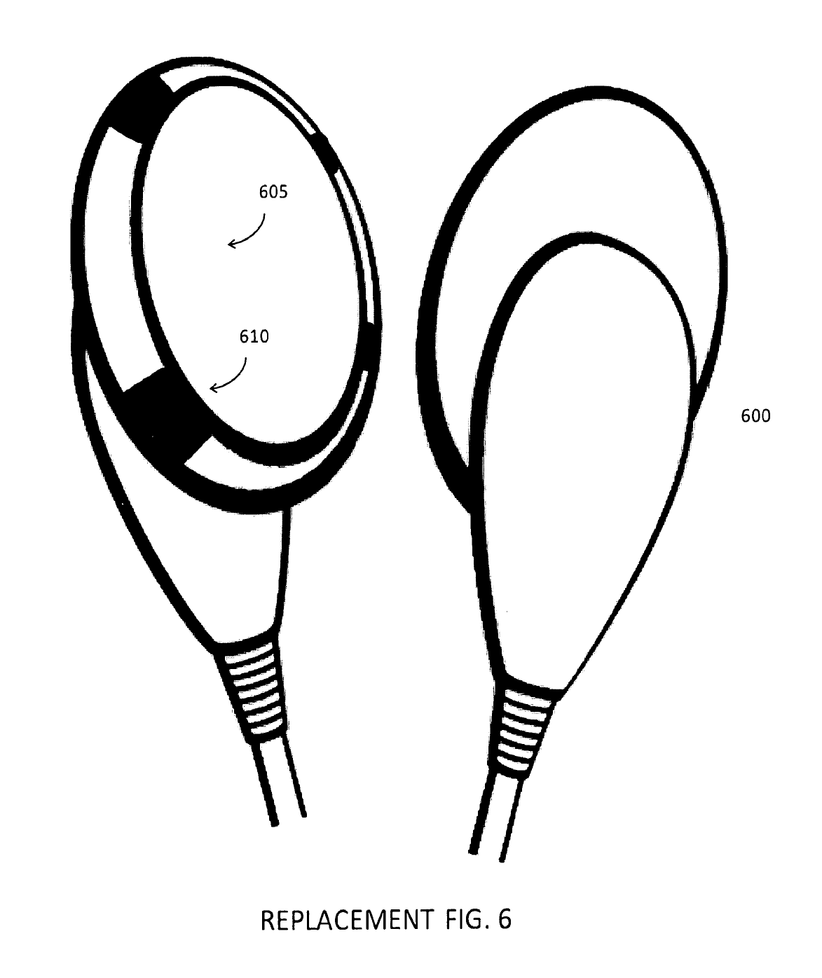

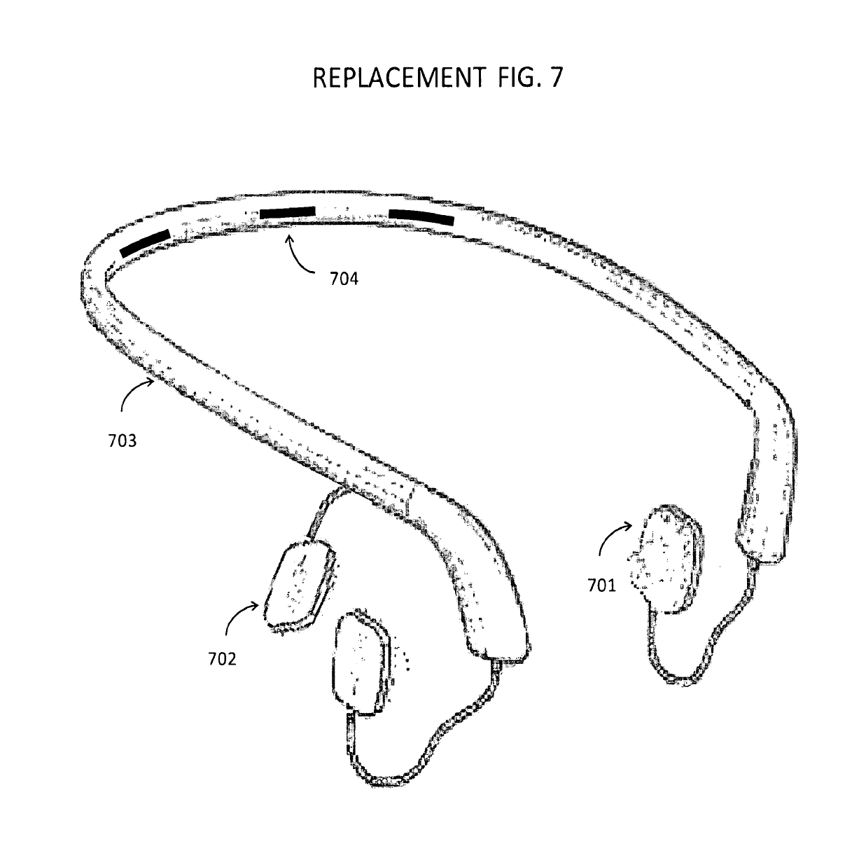

The size of the electrodes in an earphone may be a trade between being able to fit several electrodes within a confined space, and the capacitance of the electrode being proportional to the area, although the conductance of the sensor and the wiring may also contribute to the overall sensitivity of the electrodes. The ear insert may have many different shapes, the common goal for all shapes being, to have an ear insert that gives a close fit to the user's skin and is comfortable to wear, and that it should occlude the ear as little as possible. For example, FIG. 6 shows one embodiment of the invention as earphones (aka earbuds) 600, comprising an in-ear earplug having an audio speaker 605 and one or more electrodes 610. Exemplary earphones 600 sit in the concha of the ear or within the ear canal. The electrodes 610 can be positioned in the circumference of the earphone 600 or the center of the earphone 600 to make a direct contact with the skin of the concha (the outer walls or the center of the concha of the ear) or the walls of the ear canal. FIG. 7 shows an in-ear headset wherein the electrodes are placed within the ear, a ground electrode is attached to outer portion of the ear (e.g., pinna) or the neck of the user and a band that can circumnavigate the nape or other part of the neck, wherein additional bio-sensors can be placed on the band.

It is expected that one or more electrodes will be used as a ground or reference terminal (that may be attached to a part of the body, such as an ear, earlobe, neck, face, scalp, forehead, or alternatively other portions of the body such as the chest, for example) for connection to the ground plane of the device. The ground and/or reference electrode can be dedicated to one electrode, multiple electrodes or alternate between different electrodes (e.g., an electrode can alternate between ground and recording electrode).

In one embodiment, one or more electrodes can apply weak voltage/current to the subjects for neurostimulation, such as, for example, electrode arrays described in U.S. Patent Application No. 2015/0231396).

In one embodiment, the invention comprises an assembly includes one or more electrode arrays connected by one or more leads, and a neurostimulator device. For ease of illustration, the one or more electrode arrays can be described as including a single electrode array. However, through application of ordinary skill to the present teachings, embodiments may be constructed that include two or more electrode arrays that are each independent to record simultaneous EEG signals. For example, embodiments may include two, three, four, five, six, seven, eight, nine, ten, eleven, twelve, 13, 14, 15, 16, 17, 18, 19, 20, or more electrode arrays. In some embodiments, the arrays can be wired or wireless. Further, each electrode array can include one, two, three, four, five, six, seven, eight, nine, ten, eleven, twelve, 13, 14, 15, 16, 17, 18, 19, 20, 25, 30, 50, 100 or more electrodes per array. In some embodiments, the sensors can be wired or wireless.

The bio-signal data can be transmitted in any suitable manner to (and controlled by) an external device or system. In one exemplary embodiment of the present invention, the device data is transmitted to an intermediary device (e.g., client device such as a computer or mobile device) using a wired connection, such as an RS-232 serial cable, USB connector, Firewire or Lightning connector, or other suitable wired connection to transmit one or more signal. Although it is contemplated to use standard cabling, proprietary wiring with multiple parallel wires are also contemplated. Data can be transmitted in parallel or in sequence, raw or processed. The bio-signal data can also be transmitted to the intermediary device wirelessly using a wireless transmitter, e.g., an RF module. Any suitable method of wireless communication can be used to transmit the medical device data, such as a Bluetooth connection, infrared radiation, Zigbee protocol, Wibree protocol, IEEE 802.15 protocol, IEEE 802.11 protocol, IEEE 802.16 protocol, and/or ultra-wideband (UWB) protocol. The message may also be transmitted wirelessly using any suitable wireless system, such as a wireless mobile telephony network, General Packet Radio Service (GPRS) network, wireless Local Area Network (WLAN), Global System for Mobile Communications (GSM) network, Enhanced Data rates for GSM Evolution (EDGE) network, Personal Communication Service (PCS) network, Advanced Mobile Phone System (AMPS) network, Code Division Multiple Access (CDMA) network, Wideband CDMA (W-CDMA) network, Time Division-Synchronous CDMA (TD-SCDMA) network, Universal Mobile Telecommunications System (UMTS) network, Time Division Multiple Access (TDMA) network, and/or a satellite communication network. If desired, the SMART audio headphone could be transmitted to the intermediary device using both a wired and wireless connection, such as to provide a redundant means of communication, for example. Each component may have its own power supply or a central power source may supply power to one or more of the components of the device.

In various embodiments of the invention, the invention may be implemented as part of a comprehensive audio headphone system, which includes the invention headphone in communication with an intermediary device in connection or independent of a server unit. Here, it should be noticed that there is no limitation to the circuit arrangement (electric components and/or modules) between the SMART audio headphone and the external apparatus, which means the functions provided by the SMART audio headphone is flexible, for example, the acquired bio-signals can be directly transmitted to the external apparatus after digitization, or can be processed before transmission, various situations are possible. However, processing on the invention device prior to transmission can reduce the number of independent bio-signals that need to be transmitted simultaneously. Those of skill can apply techniques applied in other fields to reduce bandwidth without loss of information. Processing prior to transmission reduces the need for multiple parallel wires, reducing unwieldy cables and cost.

In one embodiment, the invention headphone can be provided with a memory to store the invention processes, the acquired bio-signals during the entire monitoring process, the music and its attributes, and the like; or the memory can be used as the buffer during wireless transmission, so that when the user is out of the receiving range of the external apparatus, the signals still can be temporarily stored for future transmission as the user is back into the receiving range; or the memory can be used to store a backup in case of poor signal quality of wireless transmission. A memory may be included in the invention headphone for data storage, and in one embodiment, the memory can be implemented as a removable memory for external access, for example, the user can take the memory rather than the whole device.

In addition, the current invention contemplates, although not necessarily requires, techniques and mechanisms for increasing the efficiency of the electrodes. For example, a single larger electrode can be replaced by several redundant smaller electrodes to reduce artifact and/or noise. In addition, high input impedance amplifier chips and active electrode approaches decrease dependency of the contact impedance. Other methods for low power consumption, high gain and low frequency response are contemplated. Further considerations for electrode design include increasing electrode biocompatibility, decreasing electrode impedance, or improving electrode interface properties through, for example, application of small voltage pulses. The invention further comtemplates incorporating novel EEG sensors with improved resolution, together with new source localization algorithms and methods for computing complexity and synchronization in signals promise continued improvement in the ability to measure subtle variations in brain function.

The schematic flowchart diagrams and/or schematic block diagrams in the figures illustrate the architecture, functionality, and operation of possible implementations of apparatuses, systems, methods and computer program products according to various embodiments of the present invention. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and computer readable program code.

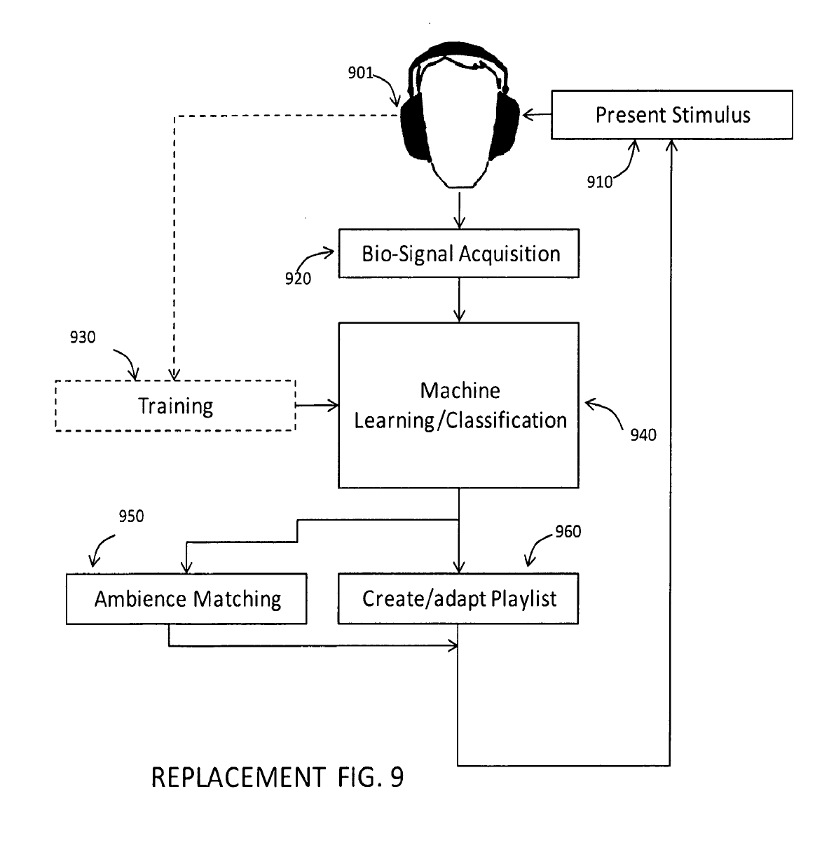

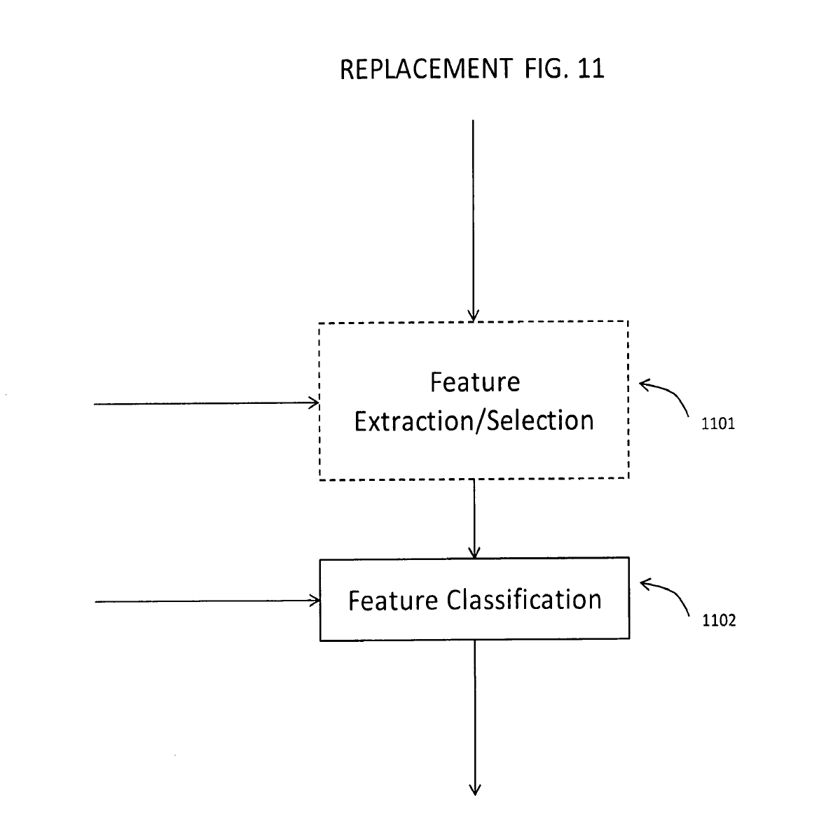

FIG. 9 illustrates an example, non-limiting system to automatically and adaptively select music that employs a machine classifier 940, such as that shown in FIG. 11, to learn and match selective physiological signals 920 to corresponding music 960. Bio-signals are acquired 920 as a feature set for the user 901 upon presentation of a stimulus 910 such as a song or other type of music. The system can be trained 930 to characterize bio-signals as particular behavior such as one or more emotions, moods and/or preferences based on a parameter values derived from pre-existing classified feature sets, user response particularly as it applies to user input, or such other methods to train the data. In addition, machine learning or pattern recognition techniques to reduce information such as feature extraction and selection techniques 1101 can be applied. User bio-signal feature set acquired from the SMART audio headphone may then be analyzed using a machine classifier 1102, a pattern classifier, and/or some other suitable technique for finding patterns in the feature set that have been determined to be associated with mood, emotion and/or preference. This information can then be used by the system to automatically create and continuously adapt the playlist of the user based on the user's state of mind. In one embodiment of the present invention, the feature set is an EEG data set reflecting an emotion, mood and/or preference of a user. An assessment of the user's behavior may be continually updated (e.g., in the behavior database) each time new EEG recordings for the user are collected and analyzed in accordance with some embodiments of the invention described herein. Training can be applied initially, periodically or continuously. This information can be stored in the behavior database (emotion/mood/preference database) for additional use, or transmitted to a client device or service to continually adapt/evolve the system or for additional functionality or analysis. In some embodiments, EEG recordings and subsequent analysis may be performed for different users and the feature output from each of the analyses may be combined into a complete feature set for a group of users.

Bio-signals can be acquired and collected using techniques and methods known in the art. In one particular embodiment, bio-signals are collected continuously, random, or periodically, for example, every few seconds. minutes, hourly and/or daily, or at different portions of a song (e.g., beginning and/or end). Acquisition can be conspicuous, or inconspicuous and discreet to the user. In one embodiment, EEG signals are acquired continuously, intermittently or periodically. In particular embodiments, specific event related potential (ERP) analyses and/or event related (power) spectral perturbations (ERSPs) are evaluated for different regions of the brain before, during and/or after a user is exposed to stimulus, or both before and each time after the user is exposed to stimulus. For example, pre-stimulus and post-stimulus differential as well as target and differential measurements of ERP time domain components at multiple regions of the brain are determined. In parallel, other physiological measurements can be acquired and correlated with measurements from the brain, for example, heartbeat or galvanic response.

Event related time, frequency and/or amplitude analysis of the differential response to assess the attention, emotion and memory retention across multiple frequency bands and locations including but not limited to (EEG measurements) theta, alpha, beta, gamma and high gamma can be assessed. In one embodiment, asymmetry indices can be calculated by manipulating information, for example, either by power subtraction or division, including user spectra of these symmetric electrode pairs.

The system may also incorporate relationship assessments using brain regional coherence measures of segments of the stimuli relevant to the entity/relationship, segment effectiveness measures synthesizing the attention, emotional engagement and memory retention estimates based on the neuro-physiological measures including time-frequency analysis of EEG measurements, and differential aural related neural signatures during segments where coupling/relationship patterns are emerging in comparison to segments with non-coupled interactions.

In one embodiment, a variety of stimuli such as music, sounds, performances, visual experiences, text, images, video, sensory experiences, or etc. can be used to elicit a physiological response. Neuro-response data or brain activity, particularly EEG, can be measured in terms of temporal, spatial, and spectral information. In addition, the techniques and mechanisms of the present invention recognize that interactions between neural regions support orchestrated and organized behavior. Attention, emotion, preference, mood, memory, and other abilities can be based on spatial, temporal, power, frequency and other related signals, including processed spectral data, but also rely on network interactions between these signals.

The techniques and mechanisms of the present invention further recognize that different frequency bands can be captured. In addition, valuations can be calibrated to each user and/or synchronized across users. In particular embodiments, templates are created for users to create a baseline for measuring pre and post stimulus differentials. According to various embodiments, stimulus generators are intelligent and adaptively modify specific parameters such as exposure length and duration for each user being analyzed.

In particular embodiments, the bio-signal collection may be synchronized with an event or time, for example with the stimulus presentation, the user's utilization of the device or on a 24-hour clock. In particular embodiments, the signal collection also includes a condition evaluation subsystem that provides auto triggers, alerts and status monitoring and components that continuously monitor the status of the user, the stimulus, signals being collected, and the data collection instruments. The condition evaluation subsystem may also present visual alerts and automatically trigger remedial actions. According to various embodiments, the invention can include data collection mechanisms or processes for not only monitoring user neuro-response to stimulus materials, but also include mechanisms for identifying and monitoring the stimulus materials. For example, data collection process may be synchronized with a music player to monitor the music played. In other examples, data collection may be directionally synchronized to monitor when a user is no longer paying attention to stimulus material. In still other examples, the data collection may receive and store stimulus material generally being presented by the user, whether the stimulus is a song, a tune, a program, a commercial, printed or digital material, an experience, audio material and the like. The data collected allows analysis of neuro-response information and correlation of the information to actual stimulus material and not mere user distractions.

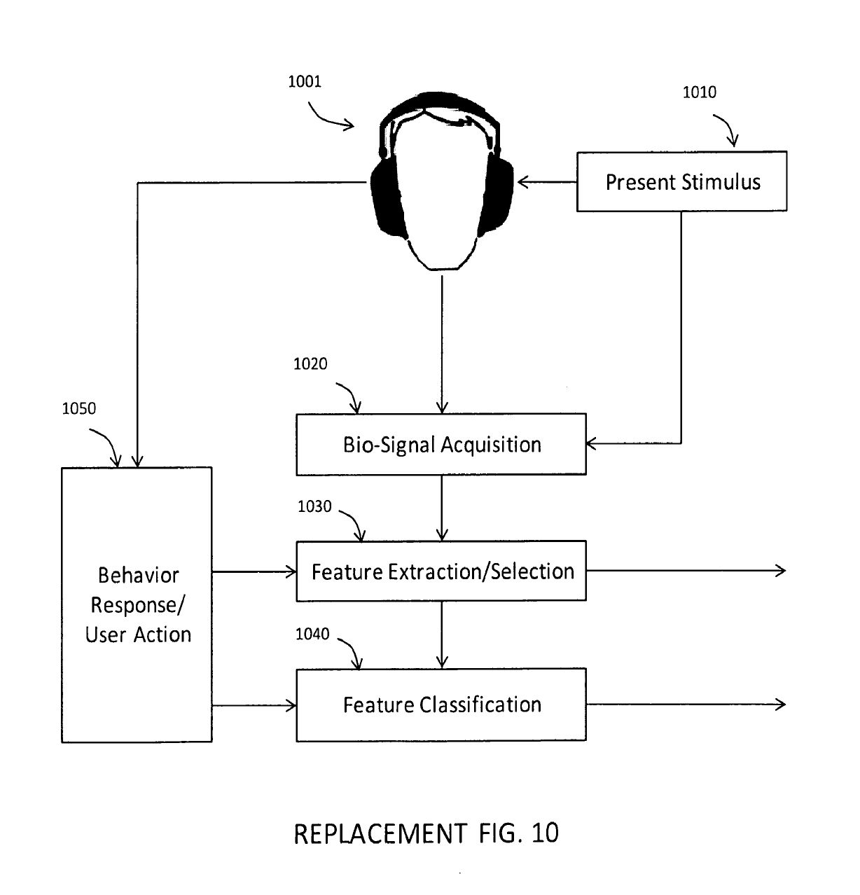

The learning system as exemplified in FIG. 9 can include automated systems with or without human intervention. For example, as shown in FIG. 10, the user 1001 can provide training guidelines 1050 such as an indication of an emotion such as happy or alertness, or preferences such as likes/dislikes of specific music thereof to initiate the training 930 of the system. In addition, the system can utilize predefined music characteristics so similar attributes such as genre or artist or characteristics of specific music (e.g., rock, jazz, pop, classical) enable classification of neuro-physiological signals and/or other physiological signals. Additional pre-defined characteristics or attributes can be provided by the user such as workout music or studying music and the like. Training 930 of such bio-signals can also include pattern recognition and object identification techniques. These sub-systems could include a hardware implementation and/or software implementations. For example, in one embodiment, classifier 1040 receives as input the complete feature set 1020 of acquired bio-signals and a database 1050 of training data. The database 1050 may include any suitable information to facilitate the classification process including, but not limited to known EEG measurements, user input, existing information regarding the stimulus, and corresponding expert evaluation and diagnosis.

In yet another embodiment, as shown in FIG. 8, one or more or a variety of modalities can be used including EEG (shown), GSR, ECG/EKG (shown), pupillary dilation, EOG, eye tracking, facial emotion encoding, reaction time, etc. User modalities such as EEG are enhanced by intelligently recognizing neural region communication pathways. Cross modality analysis can be enhanced using a synthesis and analytical blending of central nervous system, autonomic nervous system, and effector signatures. Synthesis and analysis by mechanisms such as time and phase shifting, synchronizing, correlating, and validating intra-modal determinations allow generation of a composite output characterizing the significance of various data responses to effectively perform consumer experience assessment.

The disclosed aspects in connection with a system for automatically adapting to a user's fluctuating emotions, moods and/or preferences, particularly in real life situations, can employ various A.I. (aka, artificial intelligence)—based schemes for carrying out various embodiments thereof. For example, a process for correlating bio-signals as they relate to daily emotions, moods and/or preferences swings that occur throughout the day; and/or the classifying and cataloging the characteristics of particular music as they relate to a particular preference, mood and/or emotion, and so forth, can be facilitated with the invention automatic classifier system and process. In another example, a process for cataloging EEG signals as they relate to particular music, and classifying a particular preference, mood and/or emotion to predictively create a playlist of music and or other activity, can be facilitated with the invention automatic classifier system and process, particularly, for example, as they relate to a SMART audio headphone.

FIG. 11 illustrates an exemplary, non-limiting system that employs a learning component, which can facilitate automating one or more processes in accordance with the disclosed aspects. A memory (not illustrated), a processor (not illustrated), and a feature classification component 1102, as well as other components (not illustrated) can include functionality, as more fully described herein, for example, with regard to the previous figures. A feature extraction component 1101, and/or a feature selection component 1101, of reducing the number of random variables under consideration can be utilized, although not necessarily, before performing any data classification and clustering. The objective of feature extraction is transforming the input data into the set of features of fewer dimensions. The objective of feature selection is to extract a subset of features to improve computational efficiency by removing redundant features and maintaining the informative features.

Classifier 1102 may implement any suitable machine learning or classification technique. In one embodiment, classification models can be formed using any suitable statistical classification or machine learning method that attempts to segregate bodies of data into classes based on objective parameters present in the data. Machine learning algorithms can be organized into a taxonomy based on the desired outcome of the algorithm or the type of input available during training of the machine. Supervised learning algorithms are trained on labeled examples, i.e., input where the desired output is known. The supervised learning algorithm attempts to generalize a function or mapping from inputs to outputs which can then be used speculatively to generate an output for previously unseen inputs. Unsupervised learning algorithms operate on unlabeled examples, i.e., input where the desired output is unknown. Here the objective is to discover structure in the data (e.g. through a cluster analysis), not to generalize a mapping from inputs to outputs. Semi-supervised learning combines both labeled and unlabeled examples to generate an appropriate function or classifier. Transduction, or transductive inference, tries to predict new outputs on specific and fixed (test) cases from observed, specific (training) cases. Reinforcement learning is concerned with how intelligent agents ought to act in an environment to maximize some notion of reward. The agent executes actions that cause the observable state of the environment to change. Through a sequence of actions, the agent attempts to gather knowledge about how the environment responds to its actions, and attempts to synthesize a sequence of actions that maximizes a cumulative reward. Learning to learn learns its own inductive bias based on previous experience. Developmental learning, elaborated for robot learning, generates its own sequences (also called curriculum) of learning situations to cumulatively acquire repertoires of novel skills through autonomous self-exploration and social interaction with human teachers, and using guidance mechanisms such as active learning, maturation, motor synergies, and imitation. Machine learning algorithms can also be grouped into generative models and discriminative models.

In one embodiment of the present invention, classification methods is a supervised classification, wherein training data containing examples of known categories are presented to a learning mechanism, which learns one or more sets of relationships that define each of the known classes. New data may then be applied to the learning mechanism, Which then classifies the new data using the learned relationships. In supervised learning approaches, the controller or converter of neural impulses to the device needs a detailed copy of the desired response to compute a low-level feedback for adaptation. For example, in the case of classifying one or more bio-signal markers, the desired response could be the predefined emotion, mood and/or preference, or a particular type of music such as rock or classical or jazz.

Examples of supervised classification processes include linear regression processes (e.g., multiple linear regression (MLR), partial least squares (PLS) regression and principal components regression (PCR)), binary decision trees (e.g., recursive partitioning processes such as CART), artificial neural networks such as back propagation networks, discriminant analyses (e.g., Bayesian classifier or Fischer analysis), logistic classifiers, and support vector classifiers (support vector machines). Another supervised classification method is a recursive partitioning process.

Additional examples of supervised learning algorithms include averaged one-dependence estimators (AODE), artificial neural network (e.g., backpropagation, autoencoders, Hopfield networks, Boltzmann machines and Restricted Boltzmann Machines, spiking neural networks), Bayesian statistics (e.g., Bayesian classifier), case-based. reasoning, decision trees, inductive logic programming, gaussian process regression, gene expression programming, group method of data handling (GMDH), learning automata, learning vector quantization, logistic model tree, minimum message length (decision trees, decision graphs, etc.), lazy learning, instance-based learning (e.g., nearest neighbor algorithm, analogical modeling), probably approximately correct learning (PAC) learning, ripple down rules, a knowledge acquisition methodology, symbolic machine learning algorithms, support vector machines, random forests, decision trees ensembles (e.g., bagging, boosting), ordinal classification, information fuzzy networks (IFN), conditional random field, ANOVA, linear classifiers (e.g., Fisher's linear discriminant, logistic regression, multinomial logistic regression, naive Bayes classifier, perceptron), Quadratic classifiers, k-nearest neighbor, decision trees, and Hidden Markov models.

In other embodiments, the classification models that are created can be formed using unsupervised learning methods. Unsupervised learning is an alternative that uses a data driven approach that is suitable for neural decoding without any need for an external teaching signal. Unsupervised classification can attempt to learn classifications based on similarities in the training data set, without pre-classifying the spectra from which the training data set was derived.

Approaches to unsupervised learning include:

clustering (e.g., k-means, mixture models, hierarchical clustering), (Hastie, Trevor, Robert Tibshirani, Friedman, Jerome (2009). The Elements of Statistical Learning: Data mining, Inference, and Prediction. New York: Springer. pp. 485-586)

hidden Markov models,

blind signal separation using feature extraction techniques for dimensionality reduction (e.g., principal component analysis, independent component analysis, non-negative matrix factorization, singular value decomposition) (Acharyya, Ranjan (2008); A New Approach for Blind. Source Separation of Convolutive Sources, ISBN 978-3-639-07797-1 (this book focuses on unsupervised learning with Blind Source Separation))

Among neural network models, the self-organizing map (SOM) and adaptive resonance theory (ART) are commonly used unsupervised learning algorithms. The SOM is a topographic organization in Which nearby locations in the map represent inputs with similar properties. The ART model allows the number of clusters to vary with problem size and lets the user control the degree of similarity between members of the same clusters by means of a user-defined constant called the vigilance parameter. ART networks are also used for many pattern recognition tasks, such as automatic target recognition and seismic signal processing. The first version of ART was “ART1”, developed by Carpenter and Grossberg (1988) (Carpenter, G. A. and Grossberg, S. (1988). “The ART of adaptive pattern recognition by a self-organizing neural network”. Computer 21: 77-88).

In one embodiment, a support vector machine (SVM) is an example of a classifier that can be employed. The SVM can operate by finding a hypersurface in the space of possible inputs, which the hypersurface attempts to split the triggering criteria from the non-triggering events. Intuitively, this makes the classification correct for testing data that is near, but not identical to training data. Other directed and undirected model classification approaches include, for example, naive Bayes, Bayesian networks, decision trees, neural networks, fuzzy logic models, and probabilistic classification models providing different patterns of independence can be employed. Classification as used herein also may be inclusive of statistical regression that is utilized to develop models of priority.

The disclosed aspects can employ classifiers that are explicitly trained (e.g., via user intervention or feedback, preconditioned stimuli 910 such as known emotions/moods/preferences, preexisting playlists and musical preferences, and the like) as well as implicitly trained (e.g., via observing music selection over time for a particular user, observing usage pattern (e.g., studying, working out, etc.) receiving extrinsic information, and so on), or combinations thereof. For example, SVMs can be configured via a learning or training phase within a feature classifier constructor and feature selection module. Thus, the classifier(s) can be used to automatically learn and perform a number of functions, including but not limited to learning bio-signals for particular emotions, moods and/or preferences, learning bio-signals (e.g., EEG) associated with particular music, removing noise including artifact noise, automatically categorizing music for each user based on a song's attributes, identifying song's attributes associated with personal emotions, moods and/or preferences, and so forth. The criteria can include, but is not limited to, EEG fidelity, noise artifacts, environment of the device, application of the device, preexisting information available for each music piece, song fidelity, service provider preferences and/or policies, and so on.

For example, as shown in FIG. 10, in one embodiment of the present invention, the SMART audio headphone system utilizes the intervention of the user to initiate the training of the system. User 1001 can initiate the system by (pre)selecting songs or providing general guidelines and preferences for type of music, or such other attribute, for example, user prefers a genre of music, or an artist or instrument, or a feature of a song; or pre-establishing classifications (e.g., pre-classifying) for music such as “this is a “rock” song”. Similarly, user can preselect songs that identify different guidelines and preferences based on desired use and/or application, for example, a workout, studying, concentrating, or background music. As music is played for the user, user can manually identify a preference status for each song or portion of a song (“like” or “dislike”), the emotion attributed to a song or a portion of a song (e.g., “happy” song, or “love” song”, “concentration” song, or etc.), skip or repeat a song, or such other intervention to enable the invention system to train from the bio-signals collected and acquired, in conjunction with user intervention. This system can create a feedback loop to further train and adapt the system to more precisely predict or evolve with the user's preference, mood and/or emotion.

According to various embodiments, the invention system also optionally includes a preprocessing step. Preprocessing can include steps to reduce the complexity or dimensionality of the bio-signal feature set. For example, FIG. 11 depicts the optional steps of using feature extraction and/or feature selection processes. Feature extraction techniques that exploit existing or recognized bio-signals can be applied to reduce processing but also general dimensionality reduction techniques may help, such as principal or independent component analysis, semidefinite embedding, multifactor dimensionality reduction, multilinear subspace learning, nonlinear dimensionality reduction, isomap, latent semantic analysis, partial least squares analysis, autoencoder, and the like. In addition, a feature selection step 903 can be used to select a subset of relevant features from a larger feature set to remove redundant and irrelevant features, for example reducing one or more bio-signals from a bio-signal feature set, or one or more music attributes from a music attributes feature set, or one or more emotions/moods/preferences from a emotions/moods/preferences feature set. The resulting intensity values for each sample can be analyzed using feature selection techniques including filter techniques, which can assess the relevance of features by looking at the intrinsic properties of the data; wrapper methods, which embed the model hypothesis within a feature subset search; and/or embedded techniques in which the search for an optimal set of features is built into a classifier algorithm.

In particular embodiments, the invention further comprises filters, which may or may not be part of the feature extraction/selection process, for the collected data to remove noise, artifacts, and other irrelevant or redundant data using fixed and adaptive filtering, weighted averaging, advanced component extraction (like PCA, ICA), vector and component separation methods, etc. This filter cleanses the data by removing both exogenous noise (where the source is outside the physiology of the user, e.g. RE signals, a phone ringing while a user is viewing a video) and endogenous artifacts (where the source could be neurophysiological, e.g. cardiac artifacts, muscle movements, eye blinks, etc.). The artifact removal subsystem includes mechanisms to selectively isolate and review the response data and identify epochs with time domain and/or frequency domain attributes that correspond to artifacts such as line frequency, eye blinks, and muscle movements. The artifact removal subsystem then cleanses the artifacts by either omitting these epochs, or by replacing these epoch data with an estimate based on the other clean data (fair example, an EEG nearest neighbor weighted averaging approach).

According to various embodiments, the preprocessing is implemented using hardware, firmware, and/or software. Preprocessing can be utilized prior to feature classification. It should be noted that the preprocessing like other components may have a location and functionality that varies based on system implementation. For example, some systems may not use any automated processing steps whatsoever while in other systems, may be integrated into user devices, on user client devices (computer or mobile device) or on an aggregate processing system “in the cloud”.

As shown further in FIG. 9, the present embodiment of the invention further comprises a music-matching step that matches and selects songs or other music to classified emotions/moods/preferences—represented by selected bio signals such as EEG signals. A playlist of music can be automatically created by the system in alignment with the user's manual, conscious, subconscious or emotional choice for music. Music can be stored in a music database on the device, on a stand-alone computing or mobile device, a client device, as a part of larger network or grid computing system. An identifier, for example, represented as a particular emotion, mood or preference can be associated with each song (or portions thereof) based on the bio-signals collected from the user. Identifiers can also represent the emotions/moods/preferences of multiple users (e.g., population), music attribute databases, population libraries, and the like, although, in one embodiment, identifiers are unique to the user to measure the user's immediate or real time emotion, mood and/or preference. Identifiers can be collected and aggregated, for example, in one or more databases within the system or externally, to enhance the system, to further train the system, to utilize as metadata, or other such purposes. The identifier can be temporarily or permanently associated with music, or evolving with the changing preferences of the user. For example, user can override or confirm the choice of music, which choice can be used to further train the system. In addition, identifiers can be amended or multiple identifiers can be associated with each song (or portion thereof) as the system learns to associate different emotions, moods and/or preferences to each song. For example, a “happy” song may not be manifested by the system as a happy song for that user at that particular time if played multiple times thus necessitating an alteration in the identifier, or attachment of multiple identifiers. Accordingly, the system can also associate intensity of an emotion, mood and/or preference to a particular song or music, or emotions/moods/preferences that are time or activity/environment dependent. In addition or alternatively, as described above, a playlist can be created based on the attributes of a song. For example, once a user's preference for songs are identified, the system can be utilized to discover what elements they have in common, such as the attributes of the music, to discover and create novel playlists of music.

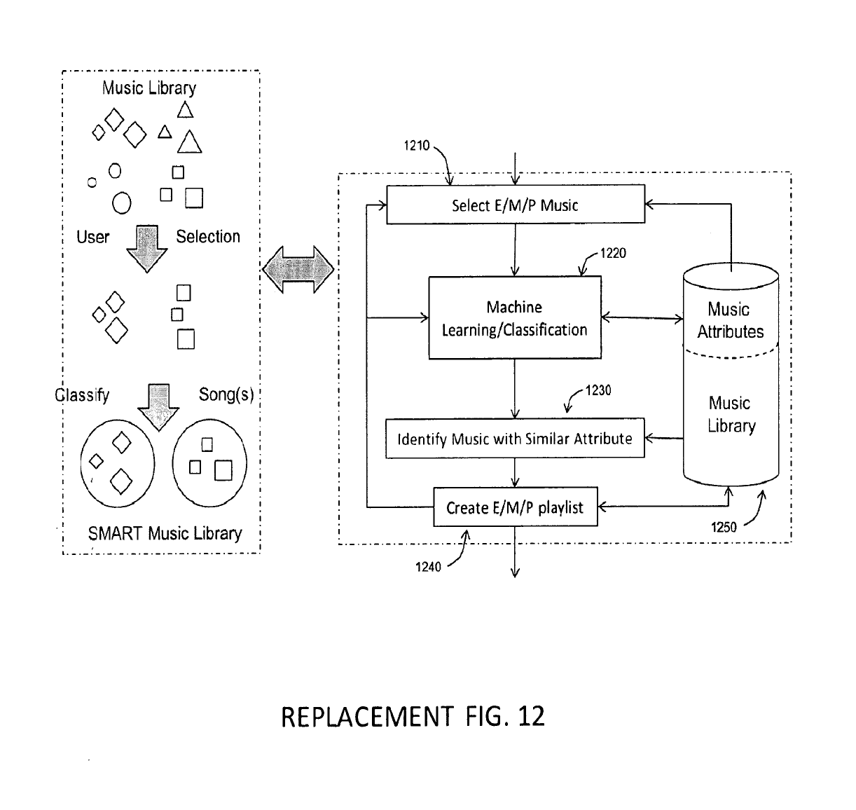

In an additional embodiment, or an alternate/independent embodiment of the invention, the system as shown in FIG. 12 comprises an audio attribute classification system to learn the attributes of music associated with a particular mood, emotion and/or preference of a user. In one embodiment, music that has been classified (e.g., by the system or by the user) for an emotion, mood and/or preference can be used to train the system, and a pattern of classified attributes generated based on similarly classified music. The attribute classification method, as described herein, may be used to create playlists of similar music (e.g., music with similarly classified attributes). The present invention can further comprise an adaptive component that continually confirms the music played and on the playlist are matched with the appropriate emotion, mood and/or preference. The classifier can learn from both matching but also non-matching music, particularly the attributes that construct that music.

In one embodiment, music selected based on attributes may be used to train a system (and, as explained further below, utilized by the system to categorize/classify music in a music database and/or identify related music) including elements or characteristics of a musical piece. Such attributes include pitch, notes within a chromatic scale, duration of a note and elements based upon duration including time signature, rhythm, pedal, attack, sustain and tempo, loudness or volume and elements based up on, pitches that lie between notes in a chromatic scale, pitches that are sampled at time intervals of fractions of a second and high resolution, harmonic key, non-musical sounds part of a musical piece or performance, voice or series of user notes occurring simultaneously with other notes, percussion, sound qualities including timber, clarity, scratchiness and electronic distortion, thematic or melodic sequences of notes, notes with sequentially harmonic roles, type of cadence including authentic, weak, amen and flatted-sixth cadences, stages of cadence, type of chord, major/minor status of a chord, notes within a chord, parts, phrases and dissonance. Attributes also include features of a song, for example genre (e.g., rock, classical, jazz, etc.), mood of a song, era the song was recorded, origin or region most associated with the artist, artist type, gender of singer(s), level of distortion (electric guitar), and the like. Libraries of attributes can be utilized, for example, Gracenote (www.gracenote.com), formerly CDDB (Compact Disc Data Base), FreeDB (http://www.freedb.org), MusicBrainz (http://musicbrainz.org), and the system utilized by Pandora (and described in “Music Genome Project” U.S. Pat. No. 7,003,515). Common attributes can be utilized to group or cluster songs, and/or to identify/label associated emotions, moods or preferences for each song.

In certain embodiment, playlists can be based on patterns which recur in more than one work can be construed as the essence of the user's preferred style. Style is inherent in recurrent patterns of the relationships between different music. The primary constituents of these patterns are the quantities and qualities captured and represented in the music database playlists, for example, pitch, duration, and temporal location in the work although other factors such as dynamics and timbre may come into play. Patterns may be discerned in vertical, simultaneous relationships, such as harmony, horizontal, time-based relationships, such as melody, as well as amplitude-based relationships (dynamics) and timbral relationships. Patterns might be identical, almost identical, identical but reversed, identical but inverted, similar but not identical, and so forth. The essence of this process is to reiteratively select the patterns of differing portions of the music and look for other instances of the same, or similar, patterns elsewhere in the database, and to compile catalogues of matching music, ranking them by frequency of occurrence, type, and degree of similarity. The objective of this search, whether the pattern-matching net is cast tightly or widely, is to detect patterns that characterize the commonalities, or “style,” of the bodies of music in the music databases unique to the emotion, mood and/or preference of the user.

From time-to-time, the present invention is described herein in terms of example environments. Description in terms of these environments is provided to allow the various features and embodiments of the invention to be portrayed in the context of an exemplary application. It will be understood that various modifications may be made to the embodiments disclosed herein. Therefore, the above description should not be construed as limiting, but merely as an exemplification of preferred embodiments. Those skilled in the art will envision other modifications within the scope and spirit of the present disclosure. Such modifications and variations are intended to come within the scope of the following claims.

For example, without limiting the present invention, the SMART audio headphone system can be utilized for a variety of applications including automatically and adaptively creating personalized playlists for a user. In addition, the device can be utilized in different environments playing not only different songs and other types of music based on the real time emotion/mood/preference of the user but also to manipulate the song and/or music depending on the application. For example, a person working out may increase the tempo of the song based on the physiological condition of the user. In one embodiment, the device can determine student (or worker) engagement and/or dis-engagement using machine learning, and modify or enhance the students engagement. Music that increases alertness can be played to modify the student's mental condition. The student engagement module, in the depicted embodiment, may be in communication with one or more students, one or more electronic learning publishers, one or more learning institutions, or the like to determine engagement of the students with regard to electronic learning material provided by the electronic learning publishers and/or the learning institutions to the students. Similarly, a person that is depressed, or stressed or prone to psychiatric, psychological or physiological anomalies such as migraines or headaches can use the device to mitigate or alleviate such conditions. In other embodiments, other actions (non-musical) can be initiated by the system, for example the invention device can be connected to network of physical objects accessed through the Internet (“Internet of Things”) to manipulate other devices or other machines (e.g., light color and brightness). Other applications include neurotraining, perceptual learning/training, neurofeedback, neurostimulation and other applications, including those that may, for example, utilize an audio stimulation,

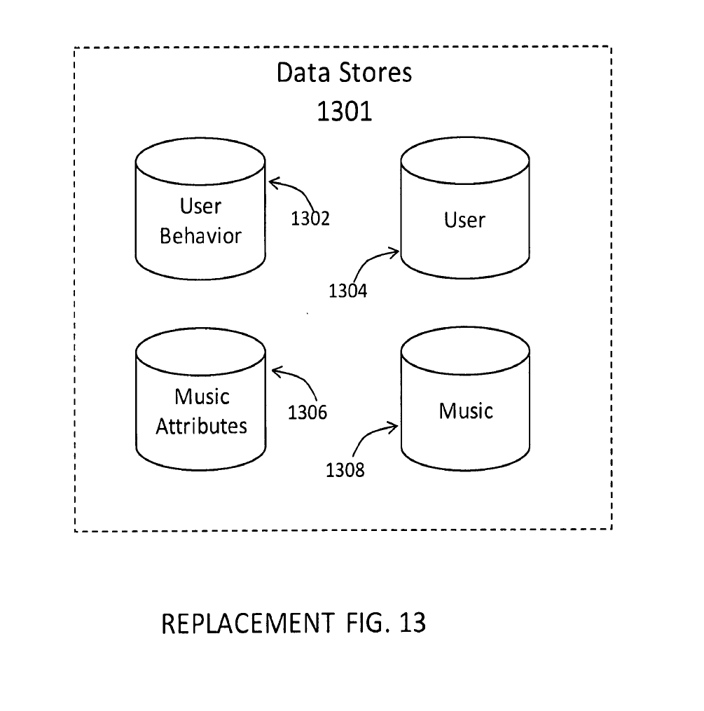

Reference is now made to FIG. 13, which is a schematic drawing illustrating exemplary data stores utilized in the present invention including a library of behavior, a library of emotions, moods and/or preferences; a library of catalogued music and/or its attributes; a user database, and a collective database of multiple users. An emotion, mood or preference library can comprise bio-signals associated with emotions, moods or preferences, for example, pre-existing libraries and/or bio-signals collected and classified by the invention system for a particular user. A music library can comprise a catalogue of music that is collected by the user or from a larger library, attributes associated with each song or music including the mood, emotion or preference of the user associated with each song or music. Music library can be stored on the device, or externally on a device or through a service. In some embodiments, the server may include a user database. The user database may comprise a database, hierarchical tree, data file, or other data structure for storing identifications or records of users, referred to generally as user records, which can be collectively stored for multiple users in the same library or in a separate collective database,.

In certain embodiment, the invention device system is configured to provide and/or allow a user to provide one or more libraries containing audio files. As used herein, a music library refers to a collection of a plurality of audio-based files. In one embodiment, invention is configured to provide an overall, or primary, library containing all the audio files stored on a device. The invention is also configured to provide, or allow, a user to create subsets, which contain two or more audio files. A library subset may contain any number of audio files, hut contains fewer than all the audio files stored on the library. The term “music library” encompasses a primary library, which contains all the audio-based files stored on electronic devices, and library subsets, which contain subsets of the audio files stored on electronic devices. A library subset may also be referred to as simply a ‘music library,”’ which may or may not be modified by another term to define or label the contents of the library, or a library subset may also be referred to as a playlist. The primary music library may refer to the entire collection of a particular audio-based file. For example, a primary library may be a primary music library containing all of the user's stored music or song files. The library subsets may be user created or created by the library application. The present invention may create library subsets based on learned emotions, moods and/or preferences associated with an audio file. For example, a song file may include attributes such as the genre, artist name, album name, and the like. The present invention may also be configured to determine various features or data associated with a library such as, for example, a library name, the date created, who created the library, the order of audio files, the date the library was edited, the order (and/or average order) in which audio files in the library are played, the number and/or average number of times an audio file is played in the library, and other such other attributes described herein, etc.

Certain aspects of the embodiments are described herein with reference to schematic flowchart diagrams and/or schematic block diagrams of methods, apparatuses, systems, and computer program products according to embodiments of the invention. It will be understood that each block of the schematic flowchart diagrams and/or schematic block diagrams, and combinations of blocks in the schematic flowchart diagrams and/or schematic block diagrams, can be implemented by computer readable program code. These computer readable program code may be provided to a processor of a general purpose computer, special purpose computer, sequencer, or other programmable data processing apparatus to produce a machine, such that the instructions, which execute via the processor of the computer or other programmable data processing apparatus, create means for implementing the functions/acts specified in the schematic flowchart diagrams and/or schematic block diagrams block or blocks.

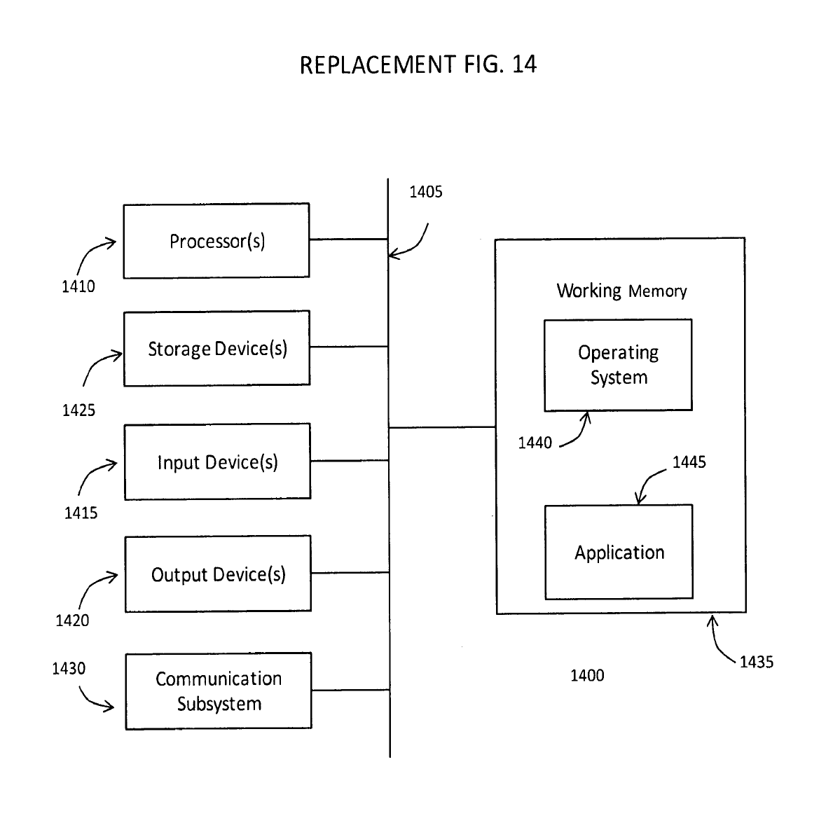

Reference is now made to FIG. 14, which is a block diagram illustrating a processing system 1300 that is able to perform the methods of FIGS. 9-12. It should be noted that FIG. 14 is meant only to provide a generalized illustration of various components, any or all of which may be utilized as appropriate. FIG. 14, therefore, broadly illustrates how user system elements may be implemented in a relatively separated or relatively more integrated manner.

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in microcode, firmware, or the like of programmable hardware devices such as field programmable gate arrays, programmable array logic, programmable logic devices or the like.

Modules may also be implemented in software for execution by various types of processors. An identified module of computer readable program code may, for instance, comprise one or more physical or logical blocks of computer instructions that may, for instance, be organized as an object, procedure, or function. Nevertheless, the executables of an identified module need not be physically located together, but may comprise disparate instructions stored in different locations which, when joined logically together, comprise the module and achieve the stated purpose for the module. In this specification, these implementations, or any other form that the invention may take, may be referred to as techniques, steps or processes.

Indeed, a module of computer readable program code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network. Where a module or portions of a module are implemented in software, the computer readable program code may be stored and/or propagated on in one or more computer readable medium(s).

The computer readable medium may be a tangible computer readable storage medium storing the computer readable program code. Any combination of one or more computer readable storage media may be utilized. A computer readable storage medium may be, for example, but not limited to, an electronic, magnetic, optical, electromagnetic, infrared, or semiconductor system, apparatus, or device, or any suitable combination of the foregoing.

More specific examples (a non-exhaustive list) of the computer readable medium may include but are not limited to a portable computer diskette, a hard disk, a random access memory (RAM), a read-only memory (ROM), an erasable programmable read-only memory (EPROM or Flash memory), a portable compact disc read-only memory (CD-ROM), a digital versatile disc (DVD), a Blu-Ray Disc (BD), an optical storage device, a magnetic storage device, a holographic storage medium, a micromechanical storage device, or any suitable combination of the foregoing. In the context of this document, a computer readable storage medium may be any tangible medium that can contain, and/or store computer readable program code for use by and/or in connection with an instruction execution system, apparatus, or device.

The computer readable medium may also be a computer readable signal medium. A computer readable signal medium may include a propagated data signal with computer readable program code embodied therein, for example, in baseband or as part of a carrier wave. Such a propagated signal may take any of a variety of forms, including, but not limited to, electrical, electro-magnetic, magnetic, optical, or any suitable combination thereof. A computer readable signal medium may be any computer readable medium that is not a computer readable storage medium and that can communicate, propagate, or transport computer readable program code for use by or in connection with an instruction execution system, apparatus, or device. Computer readable program code embodied on a computer readable signal medium may be transmitted using any appropriate medium, including but not limited to wireless, wireline, optical fiber cable, Radio Frequency (RF), or the like, or any suitable combination of the foregoing.

In one embodiment, the computer readable medium may comprise a combination of one or more computer readable storage mediums and one or more computer readable signal mediums. For example, computer readable program code may be both propagated as an electro-magnetic signal through a fibre optic cable for execution by a processor and stored on RAM storage device for execution by the processor.

Computer readable program code for carrying out operations for aspects of the present invention may be written in any combination of one or more programming languages, including an object oriented programming language such as Java, Python, Ruby, PHP, C++ or the like and conventional procedural programming languages, such as the “C” programming language or similar programming languages. The computer readable program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The computer readable program code may also be stored in a computer readable medium that can direct a computer, other programmable data processing apparatus, or other devices to function in a particular manner, such that the instructions stored in the computer readable medium produce an article of manufacture including instructions which implement the function/act specified in the schematic flowchart diagrams and/or schematic block diagrams block or blocks.

The computer readable program code may also be loaded onto a computer, other programmable data processing apparatus such as a tablet or phone, or other devices to cause a series of operational steps to be performed on the computer, other programmable apparatus or other devices to produce a computer implemented process such that the program code which execute on the computer or other programmable apparatus provide processes for implementing the functions/acts specified in the flowchart and/or block diagram block or blocks.

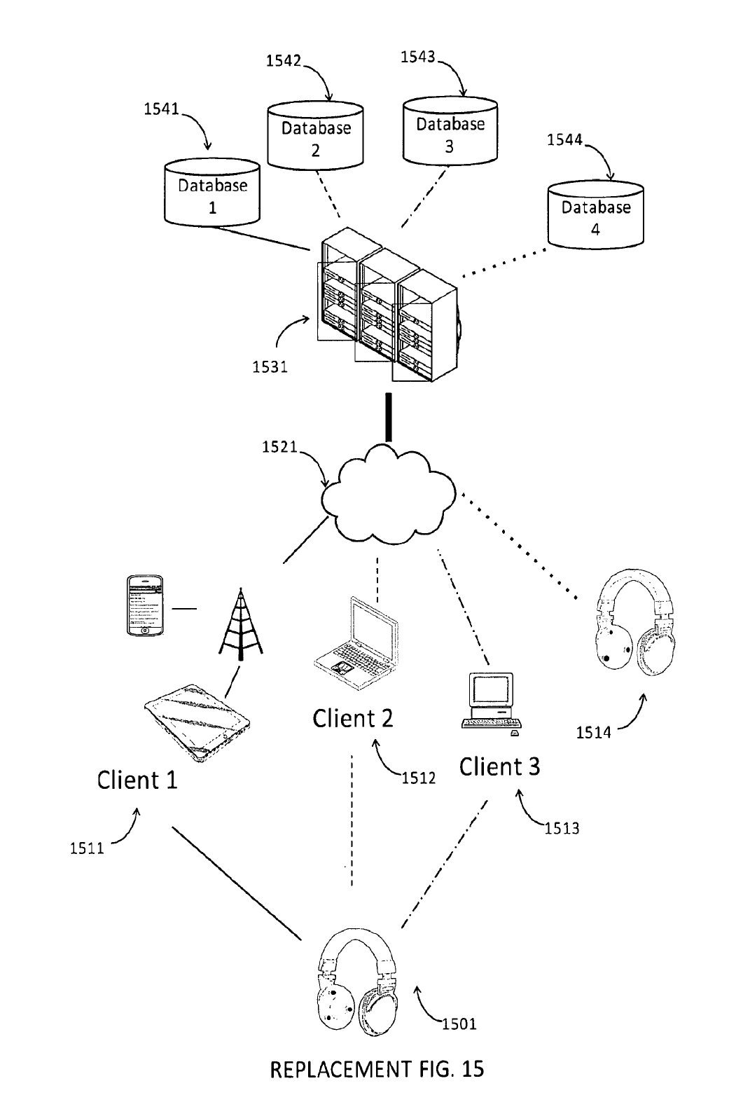

As shown in FIG. 15, certain embodiments of the invention operate in a networked environment, which can include a network. The network can be any type of network familiar to those skilled in the art that can support data communications using any of a variety of commercially available protocols, including without limitation TCP/IP, SNA, IPX, AppleTalk, and the like. Merely by way of example, the network can be a local area network (“LAN”), including without limitation an Ethernet network, a Token-Ring network and/or the like; a wide-area network (WAN); a virtual network, including without limitation a virtual private network (“VPN”); the Internet; an intranet; an extranet; a public switched telephone network (“PSTN”); an infrared network; a wireless network, including without limitation a network operating under any of the IEEE 802.11 suite of protocols, the Bluetooth™ protocol known in the art, and/or any other wireless protocol; and/or any combination of these and/or other networks.

Embodiments of the invention can include one or more server computers Which can be co-located with the headphone or client, or remotely, for example, in the “cloud”. Each of the server computers may be configured with an operating system, including without limitation any of those discussed above, as well as any commercially (or freely) available server operating systems. Each of the servers may also be running one or more applications and databases, which can be configured to provide services to the SMART audio headphone directly, one or more intermediate clients, and/or other servers.

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in applications, published applications and other publications that are herein incorporated by reference, the definition set forth in this document prevails over the definition that is incorporated herein by reference.

What is claimed is:

1. A SMART audio headphone system to adaptively and automatically select and listen to music based on learned emotions, moods and/or preferences of a user comprising an audio headphone having one or more audio speakers and one or more bio-signal sensors that adaptively acquires and classifies one or more bio-signal to learn a user's emotions, moods and/or preferences, and selects music that matches the preference, mood and/or emotion of the user.

2. The audio headphone system of claim 1 further comprising a machine classifier for acquiring and classifying physiological signals to corresponding emotions, moods and/or preferences.

3. The audio headphone system of claim 2, wherein physiological signals are acquired upon initial use by the user, intermittently, periodically, or continuously.

4. The audio headphone system of claim 2, wherein physiological signals are correlated to music.

5. The audio headphone system of claim 1, wherein the bio-signal sensors are electrodes.

6. The audio headphone system of claim 4, wherein electrodes are be positioned to read an EEG signal from the skin on the ear of the user, surrounding the ear of the user, or along the hairline around the ear or on the neck of the user.

7. The audio headphone system of claim 4, wherein headphone comprises two or more electrodes, wherein at least one least one electrode is a reference electrode.

8. The audio headphone system of claim 4, further comprising one or more earpieces supporting the speakers and the electrodes.

9. The audio headphone system of claim 4, further comprising two earpieces each supporting a speaker and one or more electrodes.

10. The audio headphone system of claim 6, wherein the earpiece comprises an earpad to support the electrode.

11. The audio headphone system of claim 1, wherein the audio headphone further comprises a headband that supports the speakers and sensors.

12. The audio headphone system of claim 6, wherein the headband comprises one or more EEG sensors.

13. The audio headphone system of claim 1, further comprising a battery and processor.

14. The audio headphone system of claim 1, further comprising an external intermediary device to store and process the bio-signals.

15. The audio headphone system of claim 1, wherein the user trains the system with user's preference, mood and/or emotion.

16. The audio headphone system of claim 2, wherein music is automatically classified and labeled based on the user's personal preferences for music, mood, or emotion.

17. The audio headphone system of claim 1, further comprising a learning mechanism to classify attributes of music based on a user's preferences, moods and/or emotions.

18. The audio headphone system of claim 1, wherein libraries of music are created based on a user's preferences, moods and/or emotions.

19. A music preference learning system comprising a learning mechanism to classify attributes of music based on user's emotions, moods and/or preferences.

20. Method of acquiring EEG signals from an audio headphone system comprising one or more electrical contact sensor and one or more speakers, wherein said method comprises:
a. Presenting a first audio stimulus such as music to a user,
b. acquiring EEG signals from the head of a user,
c. classifying the EEG signal to a user's preferences, moods and/or emotions to the audio stimulus in order to determine one or more associations between the users's emotions, moods and/or preferences and the type or attribute of music,
d. thereafter presenting additional audio stimulus similar to or different than the first audio stimulus.
